(19) United States
(12) Patent Application Publication
(10) Pub. No.: US 2019/0350150 A1
(43) Pub. Date: Nov. 21, 2019

(54) AGRICULTURAL SYSTEM AND METHOD

(71) Applicant: Avocet IP Limited, Berwick Upon Tweed, Northumberland (GB)

(72) Inventor: James Robert JENNINGS, Yarm, North Yorkshire (GB)

(21) Appl. No.: 16/466,113

(22) PCT Filed: Dec. 4, 2017
(86) PCT No.: PCT/EP2017/025350

§ 371 Date: Jun. 3, 2019

(30) Foreign Application Priority Data
Dec. 2, 2016 (GB) 1620556.9

(51) Int. Cl. A01G 31/06; A61L 2/20; A61L 2/26; C12P 5/02; C07C 29/50; C10L 1/02

(52) U.S. Cl. CPC A01G 31/06; A61L 2/202; A61L 2/26; A01K 1/01; C07C 29/50; C10L 1/02; C12P 5/023

(57) ABSTRACT

An agricultural system for generating food and fuel products includes an arrangement for supplying grain, wherein the agricultural system includes a hydroponics arrangement for receiving the supplied grain and for germinating and growing the supplied grain in a light-excluded environment in one or more hydroponics trays to generate plant growth material, and a harvesting arrangement for receiving the plant growth material and processing the plant growth material to generate corresponding livestock feed. The hydroponics arrangement includes a gaseous fumigation arrangement for gaseously fumigating the supplied grain during growing of the grain in the light-excluded environment. The gaseous fumigation arrangement is operable to employ ozone gas for fumigating the supplied grain to reduce growth of mould in the plant growth material.

### TECHNICAL FIELD

The present disclosure relates to agricultural systems and methods for producing food and fuel products. In particular, the present disclosure relates to agricultural systems for producing food and fuel products utilizing a hydroponics arrangement, and methods for using such agricultural system. Further, the present disclosure relates to computer program operable to execute aforesaid methods on the aforesaid system.

### BACKGROUND

Conventional farming systems are operable to produce food as well as by-products such as organic materials. The farming systems involve, for example, sowing of seeds into soil, and later harvesting plant products resulting from the seeds growing from the soil into corresponding plants. Moreover, the farming systems involve, for example, livestock that is fed on plant materials, and production of products derived from such livestock, for example wool from sheep, meat products, firs, skins and such like.

In specialist types of farming systems, for example in tomato production, it has become more common to employ hydroponics apparatus. Hydroponics is a subset of hydroculture, that is concerned with a method of growing plants without soil, using mineral nutrient solutions in a water solvent. The nutrients in hydroponics can be from fish waste, duck manure, or other types of nutrients. An avoidance of soil is beneficial for reducing a risk of contamination and pests present in soil, as well as more effective use of water resources. Moreover, such hydroponics employs plastics material components such as plastics material trays, plastics material sheets that can be reused and recycled, resulting in very materially efficient farming. Thus, using hydroponics is capable of providing for a very low farming ground-to-production ratio, for example based upon hydroponics apparatus disposed in a vertical configuration, whilst allowing for accurately controlled concentrations of nutrients.

When employing hydroponic to grow plants, there are potentially two different methods that are adopted:
(i) static solution cultures, wherein a given nutrient solution is employed to irrigate roots of plants; and
(ii) continuous solution cultures, wherein nutrient solutions are constantly circulated in respect of roots of plants, wherein the nutrient solutions are continuously or periodically monitored for uptake of nutrients in the nutrient solutions and corrected by additions of minerals in order maintain the nutrient solutions at a defined concentration.

Thus, in respect of static solution cultures, the nutrient solutions are changed either on a schedule, such as once per week, or when concentrations of the nutrient solutions drop below a pre-defined value, for example as determined with an electrical conductivity meter. Whenever the nutrient solutions are depleted below a certain pre-defined value, either water or fresh nutrient are added. Moreover, in respect of continuous solution cultures, nutrient solutions are arranged to flow constantly past roots of plants. A popular variation of continuous solution cultures is to employ a nutrient film technique (NFT), wherein a very shallow stream of water containing dissolved nutrients required for plant growth is recirculated past bare roots of plants in a watertight thick root mat, that develops at a bottom of a channel and has an upper surface that, although moist, is in air. Subsequent to this, an abundant supply of oxygen is provided to the roots of the plants.

An example known NFT system is based upon using elongate channels that are disposed in a substantially horizontal configuration wherein the elongate channels are tilted to have a suitable channel slope, wherein nutrient solutions are arranged in operation to flow along the elongate channels at a suitable flow rate, and the elongate channels are arranged to be of a suitable channel length. A main benefit of employing such a NFT system over other forms of hydroponics is that the plant roots are exposed to adequate supplies of water, oxygen, and nutrients. In all other forms of hydroponics production, there is a conflict between a provision of such supplies, because excessive or deficient amounts of one given type of supply results in an imbalance of one or both of other corresponding supplies. Similar design characteristics apply to all conventional NFT systems, wherein all three supplies for healthy plant growth can be met temporally simultaneously. Higher yields of high-quality produce during an extended period of cropping have been achieved. However, despite potential benefits provided by hydroponics, it is seldom employed in contemporary agricultural systems, apart from known utilization in producing specific types of crops such as tomatoes and cucumbers.

### SUMMARY

The present disclosure seeks to provide an agricultural system for generating food and fuel products. Moreover, the present disclosure seeks to provide a method of using an agricultural system for generating food and fuel products. Furthermore, the present disclosure seeks to provide a computer implementable program operable to execute aforesaid methods on the aforesaid system.

According to a first aspect of the present disclosure, there is provided an agricultural system for generating food and fuel products, wherein the system includes an arrangement for supplying grain, wherein the agricultural system includes a hydroponics arrangement for receiving the supplied grain and for germinating and growing the supplied grain in a light-excluded environment in one or more hydroponics trays to generate plant growth material, wherein the hydroponics arrangement includes a gaseous fumigation arrangement for gaseously fumigating the supplied grain during growing of the grain in the light-excluded environment, and a harvesting arrangement for receiving the plant growth material and processing the plant growth material to generate corresponding livestock feed.

The present disclosure is of advantage in that the capital costs used in the operation stated in the disclosure are very low in comparison to other processes using conventional steam reforming. In particular, the advantage of the agricultural system and the agricultural method is the efficient production of feedstock and optimal use of renewable energy in the agricultural system.

Optionally, the gaseous fumigation arrangement is operable to employ ozone gas for fumigating the supplied grain to reduce growth of mould in the plant growth material.

Optionally, the ozone gas is provided in a concentration in a range of 0.1 p.p.m.v. to 1000 p.p.m.v. (parts per million by volume at STP).

Optionally, in the hydroponics arrangement the ozone gas is provided temporally periodically for fumigating the supplied grain. More optionally, the ozone gas is used in combination with sulphur dioxide and/or chlorine gas.

Optionally, in the agricultural system the hydroponics arrangement with one of more hydroponics trays has a length-to-width ratio in a range of 3:1 to 10:1.

Optionally, in the hydroponics arrangement the one or more hydroponics trays are provided with an actuation arrangement for selectively flexing regions of the one or more hydroponics trays for sweeping ponding of nutrient solution occurring therein along a length of the one or more hydroponics trays. More optionally, the one or more hydroponics trays are arranged on a carousel for at least one of applying grain to the one or more hydroponics trays for growing the plant growth material on the one or more hydroponics trays, harvesting the plant growth material from the one or more hydroponics trays.

Optionally, the one or more hydroponics trays are arranged in one or more vertical stacks, wherein planes of the one or more hydroponics trays for receiving the grain are substantially mutually parallel.

Optionally, the agricultural system includes a feeding arrangement for providing the livestock feed to cattle. More optionally, the agricultural system includes an arrangement for receiving waste from the cattle and for anaerobically digesting the waste to generate methane gas. Moreover optionally, the agricultural system includes an arrangement for converting the methane gas to methanol fuel.

According to a second aspect, there is provided a method of operating the agricultural system pursuant to the first aspect, wherein the method includes:
(a) preparing one or more hydroponics trays of the agricultural system;
(b) distributing grain on the one or more hydroponics trays;
(c) applying a germinating solution to the grain in the one or more hydroponics trays;
(d) applying a nutrient solution to the grain to cause them to grow in light-excluded conditions;
(e) harvesting the germinated grains together with their roots; and
(f) processing the germinated grains with their roots to generate a livestock feed.

Optionally, the method includes using Spring Barley grain as the grain for distributing on the one or more hydroponics trays for cultivating to provide the livestock feed.

Optionally, the method includes fumigating the one or more hydroponics trays with gaseous ozone for reducing an occurrence of mould growth in the grain when growing on the one or more hydroponics trays. More optionally, the ozone gas is provided in a concentration in a range of 0.1 p.p.m.v. to 1000 p.p.m.v.

Optionally, in the method the ozone gas is provided temporally periodically for fumigating the supplied grain. More optionally, the ozone gas is used in combination with sulphur dioxide and/or chlorine gas.

According to a third aspect, there is provided a computer program product comprising a non-transitory computer-readable storage medium having computer-readable instructions stored thereon, the computer-readable instructions being executable by a computerized device comprising processing hardware to execute aforementioned methods.

It will be appreciated that features of the disclosure are susceptible to being combined in various combinations without departing from the scope as defined by the appended claims.

### DESCRIPTION OF THE DIAGRAMS

Embodiments of the present disclosure will now be described, by way of example only, with reference to the following diagrams wherein:

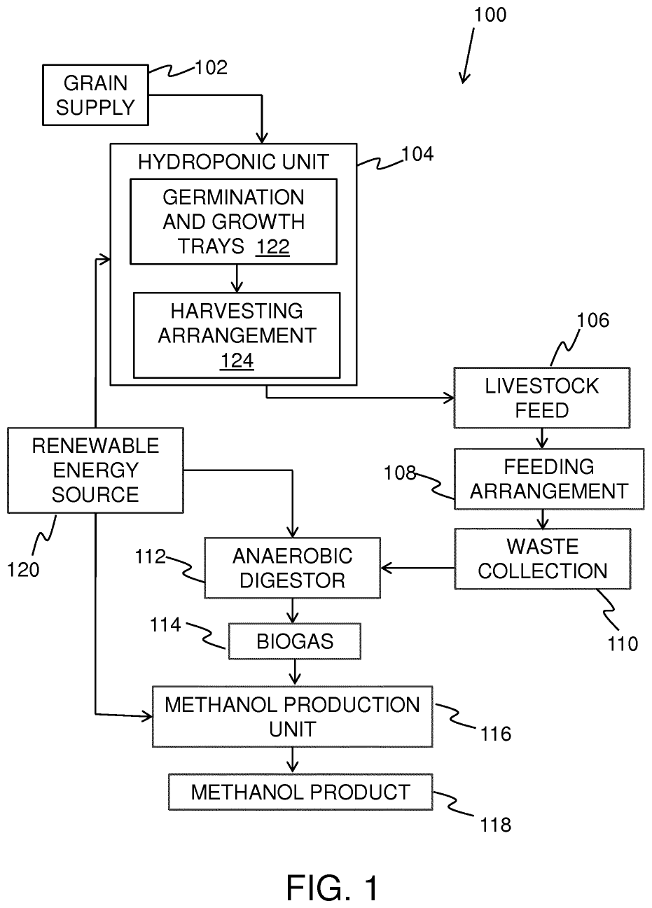

FIG. 1 is a block diagram an agricultural system, in accordance with an embodiment of the present disclosure;

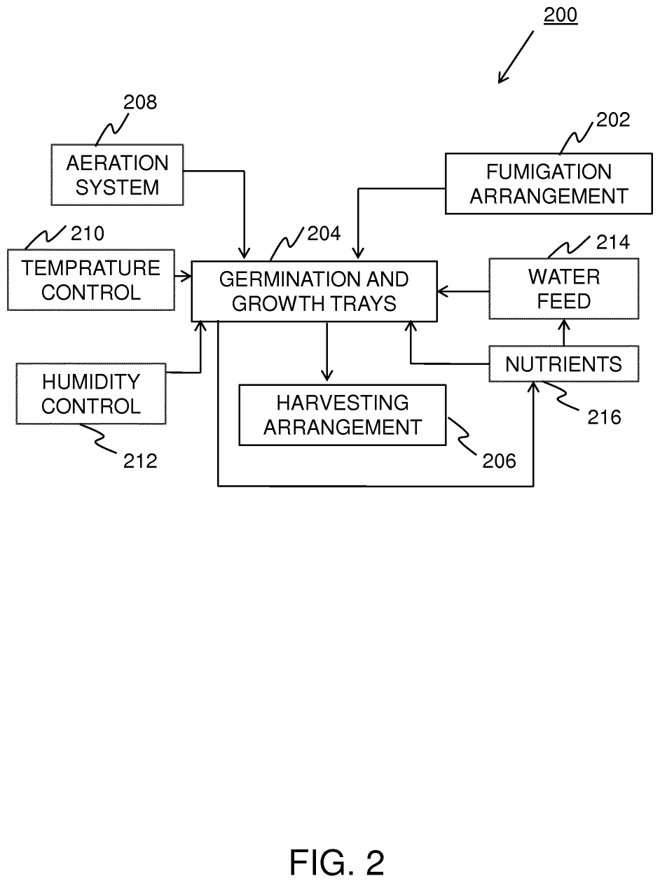

FIG. 2 is a block diagram of a hydroponics arrangement associated with the agricultural system of FIG. 1, in accordance with an embodiment of the present disclosure;

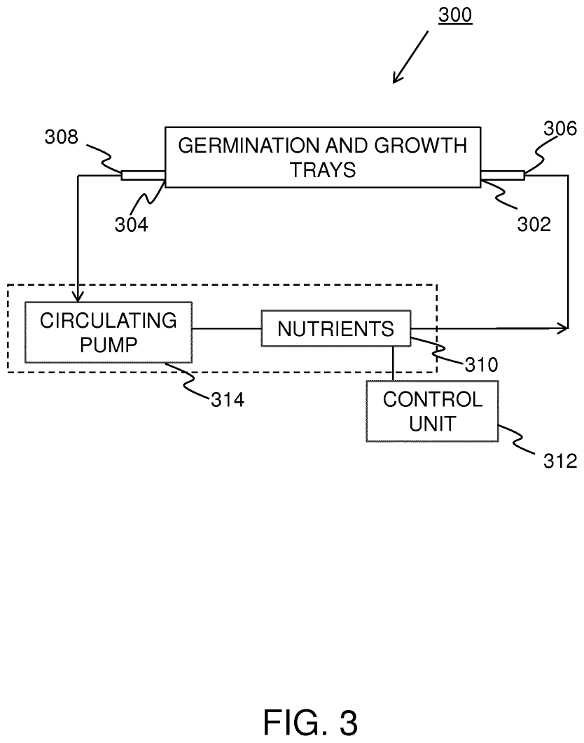

FIG. 3 is a block diagram of a germination and growth tray of the hydroponics arrangement of FIG. 2, in accordance with an embodiment of the present disclosure;

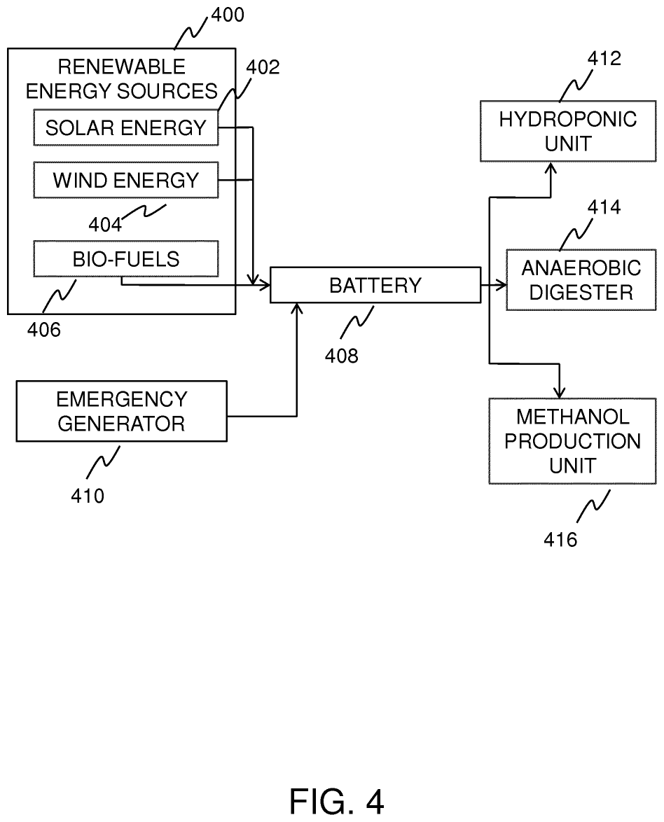

FIG. 4 is a block diagram highlighting a renewable energy of the agricultural system of FIG. 1, in accordance with an embodiment of the present disclosure; and

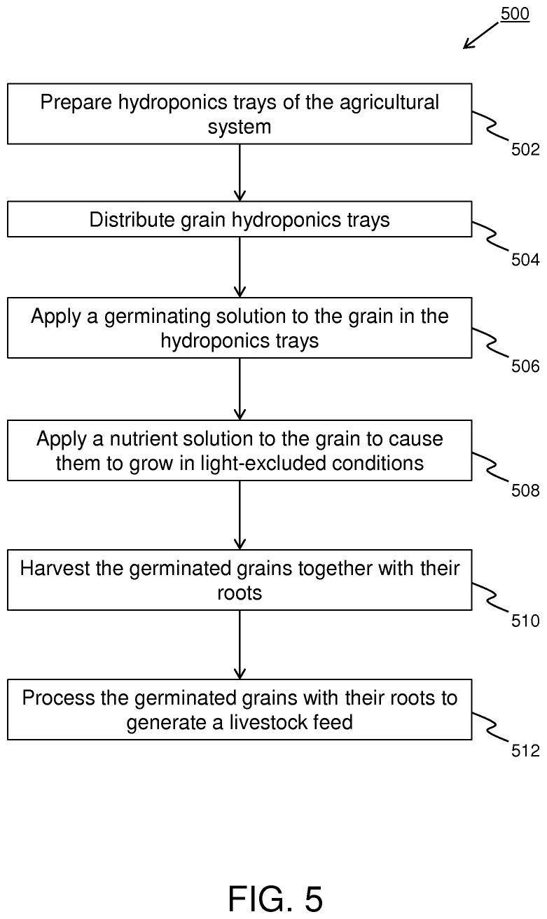

FIG. 5 is an illustration of steps of a method of using an agricultural system, in accordance with an embodiment of the present disclosure.

In the accompanying diagrams, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DESCRIPTION OF EMBODIMENTS

FIG. 1 is a block diagram an agricultural system 100, in accordance with an embodiment of the present disclosure. As shown, FIG. 1 illustrates various functional components of an agricultural system 100 for generating food and fuel products, in accordance with various embodiments of the present disclosure. The agricultural system 100 includes a grain supplying arrangement 102 for supplying grains to the agricultural system 100. The agricultural system 100 also includes a hydroponic arrangement 104 for receiving the grains from the grain supplying arrangement 102, and generating livestock feed 106 for cattle corresponding to plant growth material in the hydroponic arrangement 104. Further, the agricultural system 100 includes a feeding arrangement 108 for providing the livestock feed 106 to the cattle. The agricultural system 100 includes an arrangement 110 for receiving waste from the cattle. The waste collected in the arrangement for receiving waste 110 produces biogas 114 by anaerobic digestion in a digester 112. The agricultural system 100 further includes a methanol production unit 116 for producing methanol product 118 from the biogas 114 received from the digester 112. The agricultural system 100 also includes a renewable energy source 120 for supplying electrical energy to different components of the agricultural system 100. Thus, energy may be supplied to the hydroponics arrangement 104, the anaerobic digester 112 and the methanol production unit 116.

The agricultural system 100 pursuant to the present disclosure includes a hydroponics arrangement 104 and a livestock arrangement 106. The hydroponics arrangement/unit 104 includes germination and growth trays 122 and a harvesting arrangement 124. Optionally, there is also a more conventional grain growing arrangement for cultivating grain in an open-air environment in soil pursuant to conventional farming practice.

In an embodiment, the supplied grain from grain supplied unit 102 may be one of Spring Barley, although other types of grain such as alfalfa, maize, oats can also be used for fodder production. The supplied grain may be grown in the germination trays 104 in a light-excluded environment (for example, dark arrangement) to provide for accelerated grain germination. Growth in the dark environment causes the grain to metabolize carbohydrate sugars present in the grain, reacting with gaseous oxygen, together with water and nutrients in the hydroponic solution to provide a nourishing biological growth that is pulverized to provide feed to the aforementioned livestock 106 (for example, Piedmontese beef herd that is particularly effective at converting the feed to Omega-3 oils in their meat)

In another embodiment, the supplied unit 102 may supply grain harvested from growing plants in soil (using conventional farming techniques) and then stored in a dried state to be ready to be provided at periodic intervals into the hydroponics arrangement 104, as aforementioned, to generate a steady supply of grown plant material to the livestock 106 (i.e. growth in a light-excluded environment of the hydroponics trays 122, i.e. in a dark room).

FIG. 2 is a block diagram of a hydroponics arrangement 200 (such as the hydroponic arrangement 104 of FIG. 1) associated with the agricultural system of FIG. 1, in accordance with an embodiment of the present disclosure. As shown, FIG. 2 illustrates different functional components of the hydroponic arrangement 200 for generating livestock feed (such as the livestock feed 106 of FIG. 1) for cattle from supplied grain. The hydroponic arrangement 200 is optionally a set-up having different arrangements for controlling different growth parameters. The hydroponic arrangement 200 includes a fumigation arrangement 202 for removing microbes from the supplied grains. The hydroponic arrangement 200 also includes at least one germination and/growth trays 204 for germinating the supplied grain, and a harvesting arrangement 206 for receiving plant growth material and processing the plant growth material to generate corresponding livestock feed.

In another embodiment, the hydroponic arrangement 200 further includes an aeration system 208 for supplying fresh air to the germination trays 204, a temperature control unit 210, a humidity control unit 212, and a water feed unit 214 for continuous supply of fresh water to the germination trays 204. For example, the aeration system 208 may be a small extractor fan and vent to bring fresh air into the hydroponic arrangement 200 on periodically manner. The hydroponic arrangement 200 may be further connected to a nutrient reservoir 216 for adding a required nutritional value for germination of the supplied grain.

The water feed unit 214 of the hydroponic arrangement 200 may utilize clean water derived from solar-evaporated water vapours that is re-condensed to provide the lean water. Saline sea water, rain water runoff, river water and borehole water can beneficially be employed. Water evaporation can be achieved via evaporation from a roof arrangement including an uppermost substantially optically transparent window that transmits sunlight therethrough to an optically absorbing roof tray arrangement underneath the transparent window. The roof tray arrangement is provided with water to be cleaned and/or desalinated and sunlight-evaporated water from the roof-tray is arranged to re-condense onto the air-cooled optically transparent window that is disposed in a tilted configuration so that the re-condensed water is collected at a side of the transparent window and does not drip back into roof tray. Such an arrangement is beneficially a roof of a livestock building for accommodating the livestock.

In an embodiment, the fumigation arrangement 202 is operable to remove moulds on the supplied grain by gaseous fumigation during germination of the grain in light-excluded environment. In present embodiment in fumigation arrangement 202 may use ozone gas for fumigating the supplied grain to reduce growth of mould in the plant growth material. The ozone gas may be used periodically in combination with sulphur dioxide and/or chlorine gas for fumigating the supplied grain

In an embodiment, formation of Aspergillus on the supplied grains may be reduced by fumigating the germination and growth trays 204 by ozone gas. For example, the germination and growth trays 204 may be exposed to the ozone gas in periodic intervals. The ozone gas concentration may be in range of 0.1 p.p.m.v. to 1000 p.p.m.v. The ozone exposure may be implemented in a range 2 to 10 times per day for duration of 30 seconds to 30 minutes for each exposure. The ozone is beneficially generated from a discharge process, for example from a corona discharge process or similar. The ozone is preferably directed to roots of the germinated grain in the trays, where possible. The grain is spread evenly around the trays to avoid bunching of distribution of the grain that could cause potential occurrence of Aspergillus or similar mould growth.

Optionally in an embodiment, other gases are employed in combination with ozone, for example Sulphur Dioxide, Nitrous Oxides, to hinder mould growth, with periodic application as aforementioned. Alternatively, the gaseous treatment for Aspergillus is supplied continuously. Alternatively, the gaseous treatment for Aspergillus is selectively applied only when a trace of Aspergillus is first detected. The trays are beneficially treated with ozone and/or bleach to kill any residual Aspergillus or similar mould between growth cycles of the grain therein.

In an embodiment, the germination and growth trays 204 may be elongated in a length to width ratio in a range of 3:1 to 10:1. For example, the germination and growth trays 204 may be 30 cm to 1 meter wide. More specifically, the germination and growth trays 204 may be 60 cm to 80 cm wide. The trays 204 are optionally fabricated from a plastics material. A temperature of the trays 204 may occur in a range of 17° C. to 23° C., more optionally in a range of 20° C. to 22° C.

Optionally in an embodiment, a plurality of substantially horizontal trays 204 is arranged in a vertical stack. Optionally, the trays 204 are made of flexible solar panels. In this embodiment, the trays 204 are 5 metres long and 70 cm wide. Each tray 204 may have height in a range of 3 cm to 25 cm and the temperature of 21° C.

FIG. 3 is a block diagram germination tray 300 (such as the germination tray 204 of FIG. 2) of the hydroponic arrangement of FIG. 2, in accordance with various embodiments of the present disclosure. The trays 300 each may have a first end 302 and a second end 304, wherein the first end 302 may receive a nutrient solution input via an input pipe 306, and the second end 304 may provide a nutrient solution output via an outlet pipe 308. The input and output pipe are coupled via a nutrient solution circulating pump arrangement, comprising a nutrient reservoir 310 and circulating pump 314, and a control unit 312 for measuring characteristics of the nutrient solution (for example a pH value of the nutrient solution and its electrical conductivity) and for applying nutrients in a feedback manner to maintain the nutrient solution to have a given composition. Optionally, the nutrient solution is varied in response to growth of seeds within the trays 300. Optionally, the seeds in the trays are initially provided with a germinating nutrient solution (to trigger grain germination) followed by a growing nutrient solution to support growth of the grain.

In an embodiment, the trays 300 may be implemented on a vertically rotating carousel and may be pivotable along one of their elongate edges to enable rapid automated emptying of the trays 300 into a collection holder, whereafter the germinated grain may be pulverized as aforementioned to provide livestock feed 106. Optionally, the pulverization is only partial so as to provide the livestock feed 106 with interesting texture to munch (e.g. to provide for roughage for their digestive system).

In another embodiment, the trays 300 may be arranged at a slope of 1:100 to obtain water runoff therethrough from the input pipe 306 at the first end 302 to the output pipe 308 at the second end 304. Optionally, in different embodiments of the present disclosure the slope of the trays 300 may vary in a range of 1:50 to 1:20 for avoiding ponding. Moreover, the trays 300 may be flexible along their length and may be supported during growth of the grain therein on transverse support brackets.

Optionally, other parameters such as room temperature, cleanliness, seed treatment, amount of oxygen in the air are also key to growth. For example, small variations in temperature, could both significantly increase growth rate of the grain in the trays as well as facilitate the formation of mould which can become fatal for the whole production unit.

FIG. 4 is a block diagram of renewable energy sources 400 (such as the renewable energy source 120 of FIG. 1) of the agricultural system 100, in accordance with an embodiment of the present disclosure. The renewable energy sources 400 include at least one of solar energy 402, wind energy 404, biofuels 406, and so forth. The renewable energy sources 400 is further connected to a battery 408 for storing the electrical energy derived from the renewable energy sources 400. The battery 408 further connected to a hydroponic arrangement 412, an anaerobic digester 414, and a methanol production unit 416 (such as the methanol production unit 116 of FIG. 1) of the agricultural system 100 for supplying electrical energy therein. The battery 408 is also connected to an emergency generator 410.

In an embodiment, the renewable energy source 400 includes at least one of solar energy, wind energy, geothermal energy, biofuels, tidal energy and so forth.

In an embodiment, the renewable energy source 400 is operable to provide electrical energy required for maintaining growth parameters of the supplied grain in the hydroponic arrangement 412, digestion parameter within the digester 414 and composition adjustments for production of methanol products 118 (such as the methanol product 118 of FIG. 1) within the methanol production unit 416. For example, the renewable energy source 400 may be used for supplying electrical energy required for operating equipment controlling growth parameters of the hydroponic arrangement 412, such as heating, lighting, temperature, aeration, humidity, water feed, nutrients, and so forth.

In an embodiment, the feeding arrangement 108 of the agricultural system 100 may be operable for providing the livestock feed 106 to cattle. The livestock beneficially employs a Piedmontese beef herd, although other types of livestock may be employed.

Moreover, the agricultural system 100 may include a waste collection unit 110 for receiving waste from the cattle. The waste collection unit 110 may be connected to the digester 112 for generating the biogas 114 by converting the collected waste via anaerobic digestion. Optionally, the livestock feed 106 is fed to the cattle with foodstuffs using output growth material from the hydroponics arrangement 104 that is pulverized and supplemented with other feedstuffs to provide a wider spectrum of food components to the livestock 106.

In an embodiment, methane 114 from waste may be harvested for example from anaerobic digestion of digestive waste from the livestock 106. The methane 114 collected can be burned within the agricultural arrangement to provide energy in a methanol production unit 116, provided as gas to an external customer and/or chemically reacted with water to provide methanol fuel 118.

In an embodiment, the methane 114 produced from the digester 112 may be used as renewable energy source 400 for charging the battery 408 for supplying electrical energy to the agricultural system 100.

In an embodiment, the methanol production unit 116 may be operable to produce methanol products 118 by processing the methane 114 received from the digester 112.

In an embodiment, the anaerobic digester 112 may include employing a process of breaking down biodegradable material in absence of oxygen by using microorganisms. Such a process is contemporarily used for industrial or domestic purposes to manage waste, or to produce fuels. The processes are akin, in many respects, to fermentation that is used industrially to produce food and drink products. It will be appreciated that anaerobic digestion occurs naturally in some soils and in lake and oceanic basin sediments, where it is usually referred to as “anaerobic activity”. This is the source of marsh gas methane as discovered by a scientist Volta in year 1776.

In another embodiment, the process of breaking down biodegradable material in the anaerobic digester 112 begins with bacterial hydrolysis of input materials provided to the anaerobic digester 112, for example cattle waste as aforementioned. Insoluble organic polymers, such as carbohydrates, are broken down to soluble derivatives (including sugars and amino acids) that become available for other bacteria that are present in the anaerobic digester arrangement. Thereafter, acidogenic bacteria then convert the sugars and amino acids into carbon dioxide gas, hydrogen gas, ammonia gas and organic acids. Moreover, these acidogenic bacteria convert these resulting organic acids into acetic acid, along with additional ammonia gas, hydrogen gas, and carbon dioxide gas. Finally, methanogens convert such gaseous products to methane and carbon dioxide. Thus, such methanogens, for example methanogenic archaea populations, play an indispensable role in anaerobic wastewater treatments that are feasible to achieve using the aforementioned anaerobic digester 112.

In yet another arrangement, the anaerobic digester 112 may be operable to function as a source of renewable energy, for example for producing biogas, consisting of a mixture of methane, carbon dioxide and traces of other trace gases. This biogas can be used directly as fuel, in combined heat and power gas engines or upgraded to natural gas-quality bio-methane. There is also generated from the anaerobic digestion arrangement a nutrient-rich digestate that can be used as a fertilizer.

In an exemplary embodiment, the anaerobic digester 112 may include at least one closed vessel, for example fabricated from welded steel sheet, and is provided with a screw-feed arrangement for introducing, for example in a continuous manner, the aforementioned organic waste and/or organic crop material into the at least one closed vessel. Anaerobic digestion processes occurring within the at least one vessel result in an excess gaseous pressure to arise within the at least one vessel, wherein biogas can be selectively vented from the at least one vessel to provide biogas feedstock to a subsequent process. Beneficially, a screw-feed arrangement is used to remove digestate, for example in a continuous manner, from a lower region of the at least one vessel.

In an example, methanol synthesis is characterised by the stoichiometric ratio (H₂—CO₂)/(CO+CO₂), often referred to as the module M. A module of 2 defines a stoichiometric synthesis for formation of methanol. Further, the process is based on known chemistry but the methane is separated and purified (and steam reformed (known) using autothermal reformers to produce synthesis gas. The reactions of the synthesis process are:

CH₄+H₂O=>CH₃OH+H₂ (1)

CH₄+H₂O=>CO+3H₂ (2)

CH₄+2H₂O=>+CO₂+4H₂ (3)

Reaction (2) of catalytic methane steam reforming is strongly endothermic so that steam reforming for production of methanol requires an external heat supply. In this disclosure, the reaction can be run much closer to stoichiometric ratios, allowing more carbon content from the methane rich gas 114 (AD gas or biogas) to produce Methanol 118, thus obtaining more methanol 118 for the same amount of methane 114 input. The composition of the synthesis gas is too rich in Hydrogen for stoichiometric methanol synthesis so by adding in some of the separated CO₂ the stoichiometric ratios of methanol are balanced.

In an exemplary embodiment, the methanol production unit 116 of the system 100 may include a chemical reaction arrangement operable to provide the stoichiometric condition required for conversion of Methane to Methanol. Further, at a first stage for steam reforming in the chemical reaction arrangement the stoichiometric conditions may include but not limited to a pressure in a range of 10 Bar to 30 Bar, and a temperature in a range of 750° C. to 950° C. Furthermore, at the second stage of methanol synthesis the chemical reaction arrangement the stoichiometric conditions may include but not limited to a pressure in a range of 50 Bar to 150 Bar, and a temperature in a range of 200° C. to 250° C. In practice, use of high temperature in the first stage for steam reforming the stoichiometric conditions is advantageous in terms of higher rate of reaction and removal of impurities present in feed received from the anaerobic digestion arrangement

In another embodiment, the methanol production unit 116 may further include a methanol reformer for converting traces of Methane into Methanol received from purge stream of the chemical reaction arrangement. In this embodiment, the methanol reformer may include less exotic alloys/less active alloys as catalysts for converting traces of Methane into Methanol received from purge stream of chemical reaction arrangement. In practice, use of less exotic alloys/less active alloys as catalysts is advantageous in terms of reducing loss of methane due to recycling of the purge gasses.

In yet another embodiment, the methanol production unit 116 may be operable to receive CO₂ from an external source for improving the stoichiometric ratio of the Methane and CO₂ mixture required for adequate production of the Methanol.

It will be appreciated that both the auto-thermal reforming reaction and the steam reforming reaction require the addition of steam as a key reactive component in the reaction; however, the auto-thermal reforming reaction retains the methane as a carbon source and does not burn it externally. Hence, the oxidation products from the methane are not lost to atmosphere.

In one embodiment, the agricultural system 100 further connected to an additive source for adding an additive to the methanol product 118 to constitute a liquid fuel. In an example, the additive includes at least one of polyethylene glycol dinitrate, ammonium nitrate, urea, Avocet (Avocet is a registered trade mark in the United Kingdom) or any combination thereof. The additive source provides additive, which may be mixed with the methanol product 118 to constitute the liquid fuel, for example a diesel replacement.

Although the composition of Avocet is proprietary, and may have varied over time, the composition of the original Avocet additive includes following components as provided below:
PEG (PolyEthyleneGlycol) dinitrate—80%
Methanol—18%
Lubricity additive—1.5%
Antioxidant—0.1%

FIG. 5 is an illustration of steps of a method 500 of using an agricultural system (such as the agricultural system 100 of FIG. 1) for generating food and fuel products, in accordance with an embodiment of the present disclosure.

As mentioned above the agricultural system includes an arrangement for supplying grain (such as the arrangement for supplying grains 102) and a hydroponic arrangement (such as the hydroponic arrangement 104) for generating livestock feed (such as the livestock feed 106) for cattle.

At step 502 the flowchart 500 initiates. At the step 502, as aforementioned, one or more hydroponic trays are prepared. At step 504 the grain received from the arrangement for supplying grain are distributed on the one or more hydroponic trays. At step 506 a germinating solution is applied to the grain in the one or more hydroponic trays. At step 508 a nutrient solution is applied to the grain in hydroponic trays for generating growth in the grain in light-excluded conditions. At step 510 germinated grains are harvested with their roots. At step 512 the germinated grains with their roots are processed for generating livestock feed. The flow chart 500 terminates at step 512.

The method 500 includes the aforementioned ozone fumigation to reduce occurrence of Aspergillus and similar moulds. The method 500 includes actuation of the trays to sweep ponding along the trays to prevent formation of stagnant ponds that can result in Aspergillus or similar growth. The actuation may be achieved using an actuation arrangement whereby actuators are activated in a sequence along a length of the trays to sweep any ponding along the trays. The actuators may be electromagnetic actuators that are actuated under computer control, although hydraulic actuators can alternatively be employed.

Optionally, the present disclosure provides a computer program product comprising a non-transitory computer-readable storage medium having computer-readable instructions stored thereon, the computer-readable instructions being executable by a computerized device comprising processing hardware to execute the method of using an agricultural system for generating food and methanol products.

The agricultural system 100 of the present disclosure is advantageous on account of being susceptible to being implemented at a lower capital expenditure in comparison to known conventional systems of cattle feed and fuel production.

In addition, the agricultural system 100 of the present disclosure is highly efficient in terms of producing cattle feed (grass including seeds). For example, the agricultural system 100 described above is capable of producing the same amount of cattle feed as produced in up to 35 acres of traditional farming.

In addition, the agricultural system 100 of the present disclosure is advantageous in terms of a shorter time duration of the grain germination for harvesting plant growth material to produce livestock feed 106. For example, in agricultural system 100 described above grain germination to harvesting cycle ranges between 4 to 8 days. However, in conventional farming harvesting can be done only 2-3 times in a year.

The system and method provided in this disclosure may be configured to maintaining optimum level of CO₂ required for photosynthesis of the germinated seeds or fodder. For example, a gas feed arrangement may include one or more sensors for measuring the levels of CO₂ and/or for controlling the release of CO₂, maintaining it a required level or an optimum level of CO₂. The level of CO₂ may be controlled by a gas feed arrangement, or by adjusting one or more parameters of the fodder growth process, for example changes in temperature of operation which would with speed or slow chemical (or bio-chemical) reactions, including the fodder growth rate.

Modifications to embodiments of the disclosure described in the foregoing are possible without departing from the scope of the disclosure as defined by the accompanying claims.

Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present.

Reference to the singular is also to be construed to relate to the plural. Numerals included within parentheses in the accompanying claims are intended to assist understanding of the claims and should not be construed in any way to limit subject matter claimed by these claims.

1. An agricultural system for generating food and fuel products, wherein the system includes an arrangement for supplying grain, wherein the agricultural system includes a hydroponics arrangement for receiving the supplied grain and for germinating and growing the supplied grain in a light-excluded environment in one or more hydroponics trays to generate plant growth material, wherein the hydroponics arrangement includes a gaseous fumigation arrangement for gaseously fumigating the supplied grain during growing of the grain in the light-excluded environment, and a harvesting arrangement for receiving the plant growth material and processing the plant growth material to generate corresponding livestock feed.

2. An agricultural system of claim 1, wherein the gaseous fumigation arrangement is operable to employ ozone gas for fumigating the supplied grain to reduce growth of mould in the plant growth material.

3. An agricultural system of claim 2, wherein the ozone gas is provided in a concentration in a range of 0.1 p.p.m.v. to 1000 p.p.m.v.

4. An agricultural system of claim 2, wherein the ozone gas is provided temporally periodically for fumigating the supplied grain.

5. An agricultural system of claim 2, wherein the ozone gas is used in combination with sulphur dioxide and/or chlorine gas.

6. An agricultural system of claim 1, wherein the one of more hydroponics trays have a length-to-width ratio in a range of 3:1 to 10:1.

7. An agricultural system of claim 1, wherein one or more hydroponics trays are provided with an actuation arrangement for selectively flexing regions of the one or more hydroponics trays for sweeping ponding of nutrient solution occurring therein along a length of the one or more hydroponics trays.

8. An agricultural system of claim 1, wherein the one or more hydroponics trays are arranged on a carousel for at least one of: applying grain to the one or more hydroponics trays for growing the plant growth material on the one or more hydroponics trays, harvesting the plant growth material from the one or more hydroponics trays.

9. An agricultural system of claim 1, wherein the one or more hydroponics trays are arranged in one or more vertical stacks, wherein planes of the one or more hydroponics trays for receiving the grain are substantially mutually parallel.

10. An agricultural system of claim 1, wherein the agricultural system includes a feeding arrangement for providing the livestock feed to cattle.

11. An agricultural system of claim 10, wherein the agricultural system includes an arrangement for receiving waste from the cattle, and for anaerobically digesting the waste to generate methane gas.

12. An agricultural system of claim 11, wherein the agricultural system includes an arrangement for converting the methane gas to methanol fuel.

13. A method of using an agricultural system of claim 1, wherein the method includes:
(a) preparing one or more hydroponics trays of the agricultural system;
(b) distributing grain on the one or more hydroponics trays;
(c) applying a germinating solution to the grain in the one or more hydroponics trays;
(d) applying a nutrient solution to the grain to cause them to grow in light-excluded conditions;
(e) harvesting the germinated grains together with their roots; and
(f) processing the germinated grains with their roots to generate a livestock feed.

14. A method of claim 13, wherein the method includes using Spring Barley grain as the grain for distributing on the one or more hydroponics trays for cultivating to provide the livestock feed.

15. A method of claim 13, including fumigating the one or more hydroponics trays with gaseous ozone for reducing an occurrence of mould growth in the grain when growing on the one or more hydroponics trays.

16. A method of claim 15, wherein the ozone gas is provided in a concentration in a range of 0.1 p.p.m.v. to 1000 p.p.m.v.

17. A method of claim 15, wherein the ozone gas is provided temporally periodically for fumigating the supplied grain.

18. An agricultural system of claim 15, wherein the ozone gas is used in combination with sulphur dioxide and/or chlorine gas.

19. A computer program product comprising a non-transitory computer-readable storage medium having computer-readable instructions stored thereon, the computer-readable instructions being executable by a computerized device comprising processing hardware to execute a method of claim 13.
